(19) United States
(12) Patent Application Publication
(10) Pub. No.: US 2018/0368897 A1
(43) Pub. Date: Dec. 27, 2018

(54) RIB FIXATION DEVICE

(71) Applicant: Ledford Lee Powell, Newport Beach, CA (US)

(72) Inventor: Ledford Lee Powell, Newport Beach, CA (US)

(21) Appl. No.: 15/907,483

(22) Filed: Feb. 28, 2018

Related U.S. Application Data
(63) Continuation-in-part of application No. 15/692,057, filed on Aug. 31, 2017.
(60) Provisional application No. 62/523,670, filed on Jun. 22, 2017.

(51) Int. Cl. A61B 17/80

(52) U.S. Cl. CPC A61B 17/8076; A61B 17/8057

(57) ABSTRACT

A device for reducing and achieving fixation of a rib fracture may include a self-fixating plate having an elongate perforated band with a plurality of screw orifices extending therethrough; and at least one rib clip extending outward from an edge of the perforated band. The self-fixating plate may have an initial coiled configuration and a secondary deployed configuration, wherein the deployed configuration is less coiled curved than the initial coiled configuration.

### RELATED APPLICATION

This application claims priority to provisional patent application U.S. Ser. No. 62/523,670 filed on Jun. 22, 2017 and is a continuation in part of U.S. Ser. No. 15/692,057, filed on Aug. 31, 2017, the entire contents of which is herein incorporated by reference.

### BACKGROUND

The embodiments herein relate generally to medical devices, and more particularly, to a rib plate that allows for self-reduction and fixation along with, in some cases, a separate be attachable rib clip that can be used in conjunction with an implantable rib plate.

Over 2.1 million people are admitted each year to hospitals after a fall or accident. Rib fractures are the most common injury after a fall. Rib fractures can cause pain, days off from work, long hospital stays, and even death. Early fixation of rib fractures improves patient outcomes, meaning that patients spend less time on the ventilator, less time in the ICU, less time in the hospital, have fewer pain medication requirements, and return to work and activities of daily living sooner. However, management of rib fractures has been challenging.

In particular, the existing devices for managing and treating rib fractures have a variety of problems. First, existing treatments for rib fractures has required the use of a lot of instrumentation to reduce the fracture and apply a fixation plate. This requires large incisions for reduction and fixation. The larger the incision, the more destroyed tissue, more pain for the patient, and longer recovery times after surgery or trauma. Additionally, some existing devices violate the intercostals nerve by placing a clamp below the rib and cause excessive disruption of the intercostals muscles because of wide fixation devices or multiple prongs above and below the rubs. Moreover, the existing devices can lead to increased discomfort to the patient, which may require hardware to be removed at a second operation.

Some conventional systems have poor outcomes, because they do not have reliable and consistent results in the hands of various surgeons. Additionally, some systems lead to poor fixation, because they do not use a plate and screw technology, which is most reliable. The existing systems that do not use the plate and screw technology are more challenging to implant in the patient, which makes it less desirable to use by the surgeon.

Therefore, what is needed is a device that allows surgeons to reduce and achieve fixation of rib fractures and flails chests with a smaller and less invasive surgery, which is also easy and reproducible.

### SUMMARY

Some embodiments of the present disclosure include a device for reducing and achieving fixation of a rib fracture. The device may include a self-fixating plate having an elongate perforated band with a plurality of screw orifices extending therethrough; and at least one rib clip extending outward from an edge of the perforated band. The self-fixating plate may have an initial coiled configuration and a secondary deployed configuration, wherein the deployed configuration is less coiled curved than the initial coiled configuration.

### BRIEF DESCRIPTION OF THE FIGURES

The detailed description of some embodiments of the invention is made below with reference to the accompanying figures, wherein like numerals represent corresponding parts of the figures.

FIG. 1 is a perspective view of one embodiment of the present disclosure.

FIG. 2 is a perspective view of one embodiment of the present disclosure, shown in use.

FIG. 3 is a section view of one embodiment of the present disclosure, taken along line 3-3 in FIG. 2.

FIG. 4 is a perspective view of one embodiment of the present disclosure.

FIG. 5 is a top plan view of one embodiment of the present disclosure.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

The device of the present disclosure may be used to reduce and achieve fixation of a bone fracture, such as a rib fracture, and may comprise the following elements. This list of possible constituent elements is intended to be exemplary only, and it is not intended that this list be used to limit the device of the present application to just these elements. Persons having ordinary skill in the art relevant to the present disclosure may understand there to be equivalent elements that may be substituted within the present disclosure without changing the essential function or operation of the device.

a. Rib Clip

b. Rib Plate

c. Screws

The various elements of the device of the present disclosure may be related in the following exemplary fashion. It is not intended to limit the scope or nature of the relationships between the various elements and the following examples are presented as illustrative examples only.

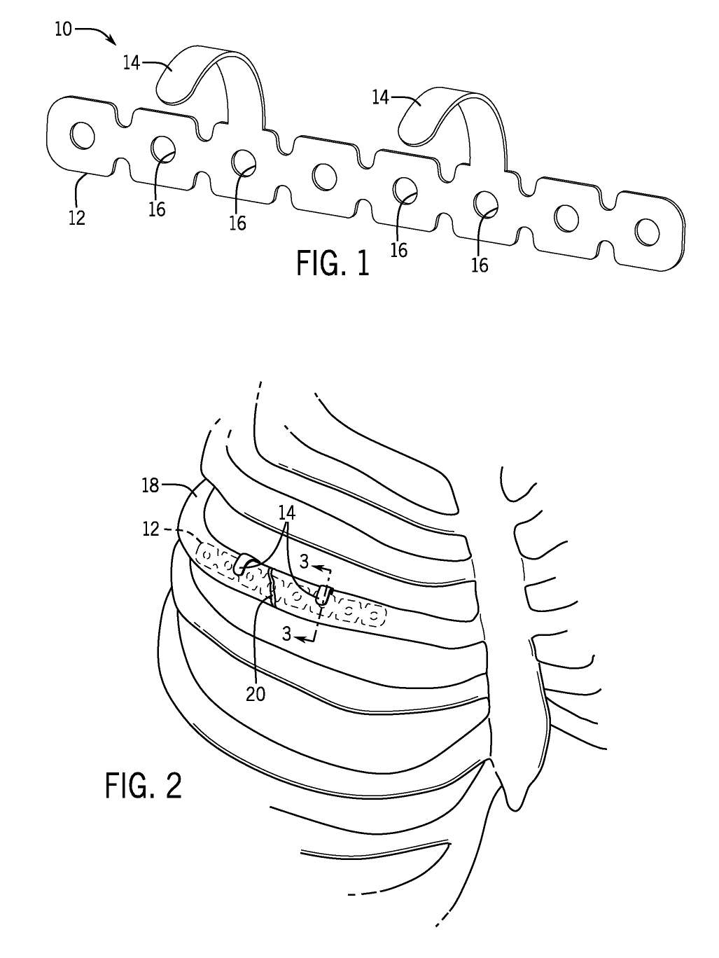

By way of example, and referring to FIGS. 1-5, some embodiments of the present disclosure include a device 10 for reducing and achieving fixation of a rib fracture 20, the device 10 comprising a rib clip 14 designed to partially encircle a rib bone 18 without obstructing the intercostals nerve, the muscle 27, or the skin 28, wherein the rib clip 14 may be partially U-shaped. The use of the rib clip 14 may eliminate the need to use a reduction clamp during the surgical procedure.

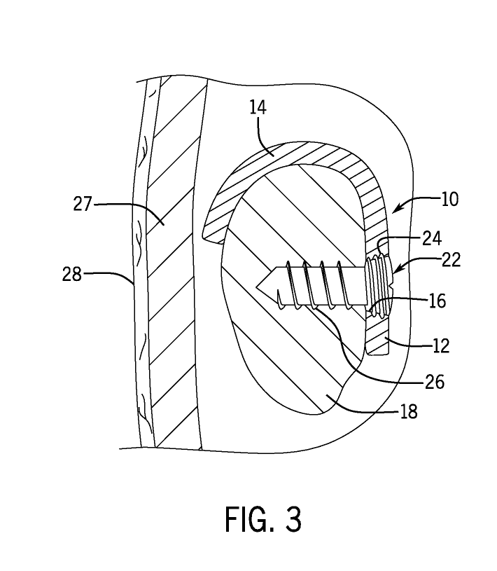

In some embodiments, the rib clip 14 may be integrated into a self-fixating plate 12, such that the self-fixating plate 12 comprises an elongate perforated band with a plurality of screw orifices 16, such as threaded screw orifices, extending therethrough, and at least one rib clip 14 extending outward from an edge of the perforated band, wherein the perforated band is positioned adjacent and parallel to the rib bone 18 over a fracture line 20, a screw 22 is screwed into the rib bone 18 through each screw orifice 16, and the at least one rib clip 14 is positioned to partially encircle the rib bone 28 without obstructing the intercostals nerve. In embodiments, and as shown in FIG. 3, the screw 22 may include a first threaded portion 24 and a second threaded portion 26, wherein the first threaded portion 24 is designed to engage with the screw orifice 16, and the second threaded portion 26 is designed to anchor the screw into the rib bone 18. Thus, the device of the present disclosure may comprise a one-piece rigid construct of a perforated band with at least one, such as two rib clips 14, incorporated into the band as a single unit.

Alternatively, some embodiments of the device include a standalone rib clip designed to engage with an existing rib plate. For example, the standalone rib clip may comprise a partially rounded U-shape with a clip screw orifice, such as a threaded clip screw orifice, at an end thereof, and the existing rib plate may comprise a plurality of plate orifices, wherein the standalone rib clip may be positioned such that the clip screw orifice aligns with one of the plurality of plate orifices and a clip screw is inserted through the clip screw orifice and the plate orifice, removably attaching the standalone rib clip to existing rib plate. Thus, the standalone rib clip may be attached to the existing rib plate at any location along a length of the existing rib plate. The combination standalone rib clip and attached existing rib plate may then be positioned on a rib bone such that the existing rib plate crosses the fracture line and the standalone rib clip partially encircles the rib bone without obstructing the intercostals nerve.

Additionally, the standalone rib clip may be used in conjunction with the self-fixating plate, such that the rib bone may be reduced and fixated with a perforated band with built in rib clips along with additional standalone rib clips attached to the perforated band via a clip screw.

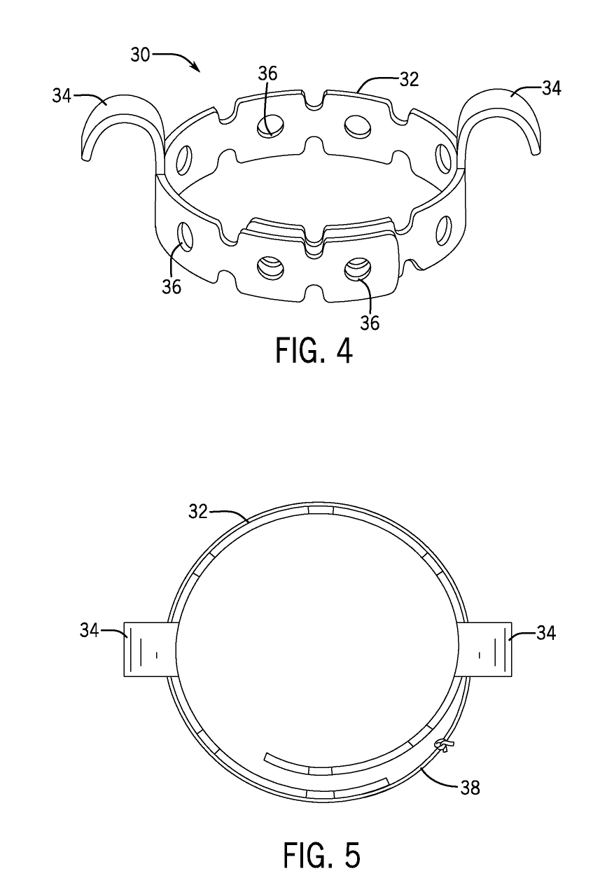

In some embodiments, and as shown, for example, in FIGS. 4 and 5, the rub fixation plate 30 may initially be in a coiled state. Thus, the plate 32 may comprise at least one rib clip 32 extending therefrom and a plurality of screw holes 36 extending therethrough. However, the plate 32 may also have a coiled construction. Moreover, a suture 38 may initially encircle the coiled plate 32 to help it maintain its coiled configuration until it is to be deployed. To use the coiled embodiment, a surgeon may insert the coiled plate 32 into the body and position it proximate to the fracture. The suture 38 may then be snipped, allowing the coiled plate 32 to be uncoiled and placed adjacent to the rib bone 18 with the rub clip(s) 32 partially encircling the rib bone 18, as shown in FIGS. 2 and 3. The plate 32 may then be secured to the rib bone 18 using at least one screw 22. The suture 38 may comprise any suitable suture material, such as a dissolvable material.

The rib clip may provide for stabilization of the plate, while the screws are used to fix the plate to the rib bone, resulting in a more reliable fixation with a less invasive approach using smaller incisions as compared to existing devices and treatment methods.

The device of the present disclosure may be made of any suitable material and, in some embodiments, comprises titanium metal. In a particular embodiment, the self-fixating plate may be about 2 mm thick and about 8 cm long. The self-fixating plate may have a gentle curve to mimic the contour of a rib bone when deployed, but may also be contoured using a device for bending the plate. The self-fixation plate may have at least two rib clips that are partially U-shaped extending from the top edge of the plate, wherein the rib clips may be malleable and approximately 1 mm thick and 5 mm wide. The standalone rib clip may be malleable and about 1 mm thick and 5 mm wide. The standalone rib clip may widen at its base to approximately 1 cm wide and may have a clip orifice, such as a threaded clip orifice, extending therethrough, wherein the clip orifice is used to screw the standalone rib clip to a plate. The clip screw may be about 3 mm long, such that it is long enough to attach the standalone rib clip to the plate but not to the rib bone. Alternatively, a longer screw may be used to attach the system to the bone. Individual devices may be sterilized and packaged in a sterile fashion.

To use the device of the present disclosure, a surgeon may position the rib clips over the top of the rib bone with at least one clip on each side of the fracture line. This will hold the two ends of the broken bone in place. The rib clips also partially grasp the upper portion of the rib bone to keep it stable for the next step. Thus, the rib clips take away the need for a reduction clamp, which is used in many operations to keep the bone edges from moving during fixation. Once the plate is applied to the broken rib bone, the plate is screwed into the broken rib bone using screws. If a rib clip is used separately form a rib plate, the surgeon may choose where the standalone rib clip will be placed on the rib plate. The rib clip is then screwed into the rib plate, and the rib plate with the newly attached rib clip is applied to the broken rib as described above.

While the device of the present disclosure is described as being used to reduce and achieve fixation of a rib bone, it may also be used to repair any broken bone in the body, wherein the shape and size of the rib clip and/or the plate may need to vary slightly to accommodate other bones.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A device for reducing and achieving fixation of a rib fracture, the device comprising:
a self-fixating plate comprising an elongate perforated band with a plurality of screw orifices extending therethrough; and
at least one rib clip extending outward from an edge of the perforated band,
wherein the self-fixating plate has an initial coiled configuration and a secondary deployed configuration, wherein the deployed configuration is less coiled curved than the initial coiled configuration.

2. The device of claim 1, further comprising a suture encircling the self-fixating plate when the self-fixating plate is in the initial coiled configuration.

3. The device of claim 2, wherein the suture comprising a dissolvable material.

4. The device of claim 1, wherein the at least one rib clip is partially U-shaped.

5. The device of claim 1, further comprising a screw designed to engage with at least one of the plurality of screw orifices.

6. The device of claim 1, wherein the screw comprises a first threaded portion designed to engage with the at least one of the plurality of screw orifices and a second threaded portion designed to engage with a rib bone.

7. The device of claim 1, wherein the rib clip comprises a titanium metal.

8. The device of claim 1, wherein the rib clip is about 1 mm thick and about 5 mm wide.

9. The device of claim 1, wherein the elongate perforated band is about 2 mm thick and about 8 cm long.

10. The device of claim 1, wherein the deployed configuration comprises a curve allowing the self-fixating plate to lay flat against a rib bone.
